(19) United States
(12) Patent Application Publication
(10) Pub. No.: US 2026/0209862 A1
(43) Pub. Date: Jul. 23, 2026

(54) HIGH SENSITIVITY ASSAY

(71) Applicant: SAGA Dx, Inc., Cambridge, MA (US)

(72) Inventors: Samuel Woodhouse, Lund (SE); Anthony Miles George, Lund (SE)

(21) Appl. No.: 19/442,697

(22) Filed: Jan. 7, 2026

Related U.S. Application Data
(60) Provisional application No. 63/743,143, filed on Jan. 8, 2025.

(51) Int. Cl. C12Q 1/6886; C12Q 1/686; C12Q 1/6869

(52) U.S. Cl. CPC C12Q 1/6886; C12Q 1/686; C12Q 1/6869

(57) ABSTRACT

The invention provides an assay that uses structural variants (SVs) as markers for tumor DNA. After tumor DNA is analyzed for tumor variants, tumor variant-specific primers are used in digital PCR to interrogate a sample from the patient for the presence of tumor DNA as an assay for MRD. Using as few as 16 SVs as tumor markers, digital PCR assays according to the disclosure have a limit of detection (LoD), or sensitivity, of less than 10 parts per million (ppm) and a specificity greater than 99.5%.

### BACKGROUND

There are various types of diagnostic tests and methods for monitoring disease. Diagnosis, screening and monitoring may involve physical exams, oral swabs, urine tests, blood tests, or medical imaging. One category of disease assay that is growing in prevalence and sophistication involves the molecular analysis of a nucleic acids. Sequences of, and mutations within, that genetic material may provide information about the presence of a disease.

For example, there are tests that look for single-nucleotide variants (SNVs) as disease indicia. When a person has cancer, it is thought that tumor DNA, which is DNA from tumor cells, may accumulate tumor specific SNVs that, if detected, could reveal the presence of the tumor. Existing cancer assays use next-generation sequencing (NGS) to sequence DNA from a subject, e.g., to detect such SNVs. However, using NGS to detect SNVs is imperfect as a test for cancer. It is understood that under some conditions, tumor DNA may escape detection when using NGS to find SNVs. Conversely, there are biological processes by which NGS may find any number of SNVs in DNA from a subject where those SNVs present no real meaningful information about a tumor.

### SUMMARY

The invention provides very sensitive assays that use structural variants (SVs) as markers for tumor DNA. Assays of the invention use digital PCR to detect a number of selected SVs markers and in which those SVs markers, and the number of those markers, gives the assay unmatched sensitivity and specificity for the detection of a tumor. To design primers for the assay, known tumor DNA is obtained from the subject and sequenced by low-pass, whole genome sequencing (lpWGS), to detect tumor-specific SVs. A subset of those tumor-specific SVs is selected and PCR primers are provided that are designed to amplify only those tumor-specific SVs. After the lpWGS and primer design, those primers may be used in a digital PCR assay to interrogate a sample from the patient for the presence of tumor DNA. When the assay is developed according to methods of the disclosure, the assay has a very low limit of detection (LoD), or sensitivity. Using fewer than a few dozen SV markers, e.g., using as few as 16 SVs as tumor markers, digital PCR assays according to the disclosure have an LoD of less than 10 parts per million (ppm). Not only is the sensitivity better than 10 ppm, the specificity is greater than 99.5%, meaning that false positives are substantially avoided.

Due to the very high sensitivity and specificity in detecting tumor DNA, assays of the disclosure are well suited to detecting small, trace amounts of tumor DNA that may be present even after a patient has undergone therapy to treat a cancer. Those amounts of tumor DNA are evidence of some small number of cancer cells remaining in the body after cancer treatment, a medical issue referred to as minimal residual disease (MRD). Using methods of the invention, MRD may detected with very high sensitivity. Thus, in the days, weeks, or years following cancer treatment, the assay may be used to reliably detect any evidence of the MRD, giving clinicians valuable information for maintaining patient health.

The described MRD assay, based on digital PCR for a limited number of selected SV markers, is well suited to detecting those markers among cell-free DNA (cfDNA) including circulating tumor DNA (ctDNA) that may be found in a patient's blood or plasma. The MRD assay may be performed on a “liquid biopsy” sample, using only a minimally-invasive blood draw. Additionally, the MRD assay has a very short turnaround time, providing a result within less than few days from the blood draw.

An MRD assay of the invention is preferably “tumor informed” in that the dPCR reagents are designed using an analysis of known tumor DNA from a tumor sample. Any suitable tumor sample may be used, and embodiments herein show the use of a formalin-fixed, paraffin embedded (FFPE) slice of tumor tissue to obtain tumor DNA. That tumor DNA may be sequenced, e.g., by NGS to generate tumor-specific sequence reads. Those sequence reads may be analyzed to detect tumor specific variants. The analysis may include mapping those reads to a reference such as one or more published human genome, or “matched normal” sequence reads obtained from non-tumor cells from the patient. Where tumor sequence reads are obtained from tumor DNA that includes the breakpoint of a structural variant (such as an inversion, duplication, translocation, deletion, insertion, or other rearrangement), those tumor reads will map to a non-tumor reference sequence with a characteristic pattern, which may be detected by software systems, such that the mapping pattern reveals a tumor-specific SV in the tumor DNA. That SV maybe used as a tumor marker going forward.

Methods of the invention may include mapping tumor sequences to a reference and discovering tumor-specific variants from that mapping. A set, aka subset, of those tumor-specific variants are selected to be used as a set of marker variants in a PCR-based assay, e.g., a digital PCR assay for MRD. Certain criteria may be applied to select the set of marker variants from all of the detected tumor variants. For example, preference may be given to: variants that exhibit a high level of duplication; driver mutations (as opposed to passenger mutations); passenger mutations; truncal mutation (e.g., that are inferred to have appeared early in the evolution of the tumor); mutations that are conserved across diverse tumor clonality; mutations for which PCR primers will not cross-reaction at distal loci; other criteria; and any combination of the foregoing. A feature of the subset of the marker variants that are used in the MRD assay is the number of marker variants that are included. It has been found that as few as sixteen (16) marker variants may be used in a digital PCR assay and while still obtaining sensitivity better than 10 ppm and specificity is greater than 99.5%. In fact, using methods of the invention, sensitivity of 0.3 ppm was obtained. Thus, methods of the invention provide for very highly sensitive and specific assays for MRD.

In certain aspects, the invention provides methods that include analyzing sequence data from tumor nucleic acid from a tumor from a subject to identify tumor-specific variants that are in the tumor nucleic acid and that are not in non-tumor nucleic acid of the subject; selecting, from among the plurality of tumor-specific variants, a set of marker variants; performing an assay to detect the marker variants in a sample from the subject; and reporting the presence of tumor DNA in the subject when the assay is positive for at least one the marker variants in the sample. A limit of detection (sensitivity) of the tumor DNA in the sample is less than 10 parts per million. Methods may include obtaining the sequence data by performing low-pass whole genome sequencing (lpWGS) on the tumor nucleic acid, wherein lpWGS means sequencing with an average coverage less than about 20× coverage, including e.g., 15× or 5× coverage. The set of marker variants may include fewer than 50 selected marker variants, e.g., fewer than 25, e.g., about 15 or 16. A specificity of the detection of the tumor DNA in the sample may be >99.5%.

The assay may be performed after the subject has been treated to remove the tumor, and the reporting may include reporting the presence of minimal residual disease (MRD) at the sensitivity <10 ppm and the specificity >99.5. The set of marker variants preferably includes fewer than 20 selected marker variants.

In some embodiments, the assay comprises digital PCR. Methods may include subjecting the sample to a pre-amplification step prior to detecting the marker variants. A pre-amplification step may include a linear amplification and the detecting of the marker variants by the digital PCR may include an exponential amplification reaction. The set of marker variants may include between 3 and 20 marker variants. The digital PCR may be used to detect greater than 3 of marker variants in two color channels at a time via radial multiplexing. The digital PCR may be performed with a limit of blank of zero and the presence of tumor DNA is reported if a positive amplification result is detected in any number of digital PCR partitions greater than zero.

Preferably, the marker variants comprise structural variants (SVs) or the breakpoints of the SVs. Methods may include—after the tumor-specific variants are identified—providing PCR primers that are specifically designed to amplify only the tumor-specific variants. The assay may comprise digital PCR performed in aqueous partitions. The aqueous partitions may comprise droplets or wells. The marker variants may comprise SVs, and the marker variants may be detected by the digital PCR using fluorescent probes. The sample from the subject may include blood or plasma obtained by a blood draw from the subject. In certain embodiments, the limit of detection (sensitivity) of the tumor DNA in the sample is 0.3 ppm.

Aspects of the invention provide methods for detecting minimal residual disease (MRD) comprising: performing an MRD assay on a sample from a subject who has undergone cancer treatment to detect a set of selected marker variants that are in tumor nucleic acid and not in non-tumor nucleic acid of the subject; and reporting the presence of MRD in the subject when the assay is positive for at least one the marker variants in the sample, wherein the MRD is detected with sensitivity <10 ppm, and specificity >99.5%. The sensitivity and specificity may be achieved using fewer than thirty (e.g., 16) of the marker variants.

The MRD assay may use digital PCR. Methods may include subjecting the sample to a pre-amplification step prior to detecting the marker variants. The pre-amplification step may include a linear amplification and the detecting of the marker variants by the digital PCR may include an exponential amplification reaction.

In some embodiments, the set of marker variants includes between 3 and 20 marker variants, and the digital PCR is used to detect greater than three of marker variants in two color channels at a time via radial multiplexing. The digital PCR may be performed with a limit of blank of zero and MRD may be reported if a positive amplification result is detected in any number of digital PCR partitions, e.g., any number greater than zero, even just one. The marker variants are preferably structural variants (SVs) or the breakpoints of the SVs. The MRD assay may use PCR primers that are specifically designed to amplify only tumor-specific variants.

In certain embodiments, the assay comprises digital PCR performed in aqueous partitions, in which the aqueous partitions are, e.g., droplets or wells, the marker variants comprise SVs, and the marker variants are detected by the digital PCR using fluorescent probes such as fluorescent hydrolysis probes or molecular beacons. The sample from the subject may include blood or plasma obtained by a blood draw from the subject and the marker variant may be found among cell-free nucleic acid in the blood or plasma.

### DETAILED DESCRIPTION

The invention provides methods that are useful to detect a tumor in a subject using a detection assay. In certain aspects, the invention provides methods that include analyzing sequence data from tumor nucleic acid from a tumor from a subject to identify tumor-specific variants that are in the tumor nucleic acid and that are not in non-tumor nucleic acid of the subject, selecting, from among the plurality of tumor-specific variants, a set of marker variants; performing an assay to detect the marker variants in a sample from the subject; and reporting the presence of tumor DNA in the subject when the assay is positive for at least one the marker variants in the sample, wherein a limit of detection (sensitivity) of the tumor DNA in the sample is less than 10 parts per million. Methods may include obtaining the sequence data by performing low-pass whole genome sequencing (lpWGS) on the tumor nucleic acid, wherein lpWGS means sequencing with an average coverage less than about 20× coverage. The set of marker variants may include fewer than 50 (e.g., 16) selected marker variants. A specificity of the assay may be >99.5%. The assay may detect minimal residual disease (MRD) with sensitivity <10 ppm and specificity >99.5.

The initial sequence data analysis, which may involve next-generation sequencing (NGS) of a tumor sample such as from a biopsy or a formalin-fixed, paraffin embedded (FFPE) tumor slice, and may proceed by low-pass, whole genome sequencing (LP-WGS), may be performed at one point in time to detect the plurality of tumor-specific variants. Those variants are analyzed to select a set of marker variants.

In various embodiments described herein, the sequence data may be obtained by sequencing DNA from an FFPE slice of the tumor; a library preparation protocol tailored to FFPE-sourced nucleic acid may be used; the tumor nucleic acid may be sequenced by LP-WGS; a computer system may be used to detect and rank tumor SVs and select a marker variant and to design primers specific for the marker variant; the primer pair may be used in a detection assay for a subject that has undergone treatment to eradicate the tumor; the sample may be a blood draw liquid biopsy, the detection assay may involve digital PCR with the sample in aqueous partitions using an amplification reaction and fluorescent hydrolysis probes; detecting fluorescence from the aqueous droplets may indicate the presence of the tumor nucleic acid in the sample; and/or the assay may be performed to detect minimal residual disease after the treatment.

### FFPE DNA Extraction

Methods of the disclosure may include obtaining nucleic acid from a formalin-fixed, paraffin embedded slice of a tumor, so that the tumor nucleic acid may be sequenced. Tissue obtained by biopsy or surgery for pathological examination may be fixed in a fixative, such as formalin and embedded in paraffin, yielding formalin fixed, paraffin embedded (FFPE) blocks. Small (e.g., a few micrometer-thick) sections may be sliced from the blocks and stained on slides for microscopic analysis. Such slides are typically retained as a pathology archive.

Methods herein may use protocols for extracting DNA from FFPE samples and preparing high-quality sequencing libraries from the FFPE-extracted DNA. To extract nucleic acid, the sample is loaded into a tube such as microcentrifuge tube. A tissue lysis buffer and proteinase K (PK) solution mix may be added to the tube. Steps of protocols herein may be performed using reagents and material sold under the product name truXTRAC FFPE total NA (tNA) Ultra Kit by Covaris. The FFPE sample may be immersed in the tissue lysis buffer/PK solution mix and sonicated in a ultrasonication instrument according to manufacturer instructions for paraffin emulsification. The steps may be performed in laboratory test tubes, wells of a plate, microcentrifuge tubes, or tubes in a multi-tube strip.

After the tube is collect, it is centrifuged, e.g., spun at 5 k g for about 15 minutes, to form a pellet that includes DNA. The described protocols provide high quality DNA, suitable or sequencing, with high yield from FFPE tissue samples. Preferably, the pellet is rehydrated with a suitable buffer such as buffer BE from Covaris and more preferably a tissue lysis buffer/PK solution mix is used. The tube may be sonicated to resuspend material of the pellet, and optionally treated with RNase. A DNA purification column may be placed into a collection tube. The sample is transferred into the column and the tube spun. Following DNA purification protocol instructions, the column is washed with buffer(s) such as BW Buffer and B5 Buffer (Covaris). Finally, the column is eluted with an elution buffer, eluting the DNA from the column. The collected (eluted) DNA may be analyzed or stored long-term. Methods of the disclosure produce high quality and high yield sequencing libraries from FFPE-extracted DNA.

### Library Preparation

Having extracted DNA from a sample, methods may include library preparation, which generally includes fragmentation, adaptor ligation, and amplification. When the source is a tumor biopsy, nucleic acids in very small quantities, or preserved (e.g., FFPE) sample, extracted DNA may be fragmented via a fragmentation step that may be more gentle and less damaging than conventional protocols. In some embodiments, the eluate that includes the extracted DNA is sheared or fragmented to yield fragments with an average fragment size of at least about 800 base-pairs. Any suitable approach may be used for shearing including enzymatic shearing, nebulization, sonication, Covaris shearing, or others. In some embodiments, it may be preferable to produce fragments that have an average size with a peak approximately within the range of about 500, preferably at least about 600 or 700, and most preferably at least about 800 base pairs (bp) to 1,000 bp. A cocktail of restriction enzymes may be composed that will, on average, cut genomic DNA on about 800 to 1,000 base intervals. Preferred embodiments use a sonicator or adaptive acoustic focusing (AFA) instrument (Covaris). Embodiments may use a Qubit instrument to evaluate quantity and/or a TAPESTATION automatic electrophoresis instrument to evaluate fragment length, using manufacturer's literature for guidelines for the sonication instrument. One approach is to shear a very small sample to the desired optical density to establish the instrument settings to be used for the bulk of the sample. The resultant shearing protocol produces 800 to 1000 base fragments.

The fragments may be repaired enzymatically. Enzymatic repair on such long fragments can correct specific injuries associated with FFPE storage and handling. Preferably the fragments are treated with enzymes such as DNA glycolase, an apurinic/apyrimidinic (AP) endonuclease, DNA polymerase, and/or ligase. DNA repair enzymes and Structure-specific endonucleases are enzymes that cleave DNA at a specific DNA lesion or structure. Those enzymes can be used for repair of DNA from sample degradation due to oxidative damage, UV radiation, ionizing radiation, mechanical shearing, formalin fixation (post extraction) or long-term storage. Those enzymes may perform any combination of base excision repair (BER), DNA mismatch repair, nucleotide excision repair, elimination or repair of large DNA secondary structures using T7 Endonuclease I, nick elimination (ligation), and others.

Preferably end repair is performed, which can be understood as a separate step or as included in enzymatic repair. End repair may use reagents such as the SureSelect XT Library Pep Kit ILM from Agilent or the IDT xGen cfDNA & FFPE Library Preparation Kit, performed in a thermocycler, e.g., as described in Agilent, 2021, SureSelectXT Target Enrichment System for the Illumina Platform, Protocol, Manual part number G7530-900000 by Agilent Technologies, Inc. (102 pages), or as described in IDT, 2022, xGen cfDNA & FFPE DNA Library Prep v2 MC by Integrated DNA Technologies (18 pages), both incorporated by reference.

In some embodiments, the end-repaired fragments are purified using magnetic beads and a magnetic separation device. A bead to DNA fragment ratio of about 0.7× may be used. That ratio of beads (e.g., about 45 μL AMPure XP beads to about 100 μL end-repaired DNA sample) is mixed, incubated, and placed on a magnetic stand. Due to ingredients in the bead mixture (e.g., PEG) the charged DNA backbone holds DNA to the beads. One feature of embodiments of the disclosure may be a minimal or low-bead ratio, which, in combination with the fragment length and subsequent steps, provides high quality, high-yield sequencing libraries from FFPE samples. Enzymes or other reagents may be washed away, and DNA may be eluted into a ligation mix.

Methods may include ligating adaptors to the fragments to form adaptor-ligated fragments. Any suitable approach may be used. Some embodiments include dA tailing at the 3′ ends of the fragments (e.g., using a dA-tailing master mix, e.g., from Agilent) and ligating suitable adaptors. Optionally, a bead cleanup step like above may be performed between dA tailing and ligation. Preferred embodiments add paired-end or Illumina Y adaptors. One kit and protocol well suited for use within this protocol is the xGen cfDNA & FFPE DNA Library Prep Kit sold by Integrated DNA Technologies, Inc. (Coralville, IA). The adaptor ligated fragments may be subject to a size-selection step to isolate selected adaptor-ligated fragments with an average size within a range of about 500 to about 1000 base-pairs from unwanted material. More specifically, preferred embodiments use a tight size selection for fragments in the range of about 550 to about 900 bp.

The selected adaptor-ligated fragments may be amplified to obtain amplicons. The PCR input is combined with PCR reaction mix (primers, buffer, dNTP, polymerase) typically according to instructions from a reagent vendor. E.g., 35 μL PCR reaction mix with 15 μL PCR input. The tube is thermocycled. In most cases, five cycles will produce adequate yield at this stage. The result is a plurality of clonal amplicons copied from nucleic acid in a tumor sample. The amplicons may have sequencing adaptors or any suitable primer binding sites at either or both ends. At this stage, a library preparation is complete.

The described extraction and library preparation protocols may be optimized, compared to commercially available kits and protocols, to compensate for damage that is characteristic of FFPE samples and their extraction. For example, after emulsification of the paraffin, DNA may be subject to a limited fragmentation process designed to only fragment the DNA to a large peak length not found in existing protocols. After enzymatic repair, the fragments are subject to a gentle bead cleanup with only a fraction of a quantity of beads found in commercial protocols. The resultant fragments are subject to adaptor ligation and an extra purification with size-selection step is performed on the adaptor-ligated fragments prior to amplification. Each of the steps—limited fragmentation, gentle bead clean-up, and purification after adaptor ligation with size-selection step—may contribute importantly to the preparation of high-quality sequencing libraries from FFPE samples.

Because protocols of the invention are useful to prepare high-quality sequencing libraries from FFPE tissue, they are useful for discovering tumor-specific mutations (e.g., structural variants) when applied to FFPE tumor samples, such as from a tumor biopsy. Once a tumor-specific somatic structural variant is known and described, that variant may be used subsequently as a marker for the presence of that tumor. In fact, protocols for library preparation from FFPE tumor samples are designed to yield, and have been found to yield, sequencing libraries of sufficient quality to identify somatic variants even without so-called “matched normal” DNA sequences from the same patient. Instead, tumor DNA may be extracted from an FFPE tumor sample according to protocols described herein, sequenced, and analyzed to identify putative structural variants (SVs). Algorithms are then applied to exclude artifacts of sample-handling and to compare the remaining putative SVs to references and/or databases to filter out germline SVs. Such an analysis may provide an identification of tumor-specific somatic SVs that are present in a patient's tumor DNA. That information is then used to design reagents to assay future samples from the patient for those same tumor-specific somatic SVs. For example, an informatics pipeline may be used to design amplification primers and fluorescent probes for the detection of such variants by a digital PCR assay. Some embodiments identify tumor-specific SVs present in a patient's tumor DNA and then use an informatics pipeline to design primers and fluorescent probes useful for detecting by digital PCR those SVs in cell-free tumor DNA in blood or plasma, e.g., from a liquid biopsy.

### Sequencing

Nucleic acid obtained according to methods of the disclosure is preferably sequenced to obtain sequence data. For example, methods may include sequencing DNA from a tumor sample from the subject to obtain sequence reads.

Sequencing may be by any method known in the art. Suitable DNA sequencing techniques may include the dideoxy chain-termination sequencing technique known in the art as Sanger sequencing, which uses labeled terminators and gel separation in a slab or capillary. Sequencing may include the sequencing by synthesis using reversibly terminated nucleotides and the detection of pyrophosphate in the technique known as pyrosequencing commercialized by ROCHE 454. Sequencing may proceed by techniques that include allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Separated molecules may be sequenced by sequential or single extension reactions using polymerases or ligases as well as by single or sequential differential hybridizations with libraries of probes. Sequencing may be performed using one of the single molecule, long read sequencing platforms commercialized by HELICOS, PACIFIC BIOSCIENCES, or OXFORD NANOPORE.

Sequencing techniques and instruments that may be used include, for example, those offered by ILLUMINA, INC. or ULTIMA GENOMICS. Illumina sequencing is based on the amplification of a sequencing library described above on a solid surface of a flow cell using fold-back PCR and anchored primers. Amplicons of adaptor-ligated fragments that constitute the sequencing library are annealed to oligos attached to the surface of flow cell channels that are extended by which the amplicons are bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured, and the identity of the first base is recorded. The 3′terminators and fluorophores from each incorporated base are removed and the incorporation, detection and identification steps are repeated. Sequencing according to this technology is described in U.S. Pat. Nos. 7,960,120; 7,835,871; 7,232,656; 7,598,035; 6,911,345; 6,833,246; 6,828,100; 6,306,597; 6,210,891; U.S. Pub. 2011/0009278; U.S. Pub. 2007/0114362; U.S. Pub. 2006/0292611; and U.S. Pub. 2006/0024681, each of which are incorporated by reference in their entirety.

Sequencing generates sequence data and for short-read, ensemble sequencing platforms such as the ILLUMINA platform, the sequence data comprises a large number of short sequencing reads typically accessible from the ILLUMINA system in a computer file format known as FASTQ.

The sequencing instrument and technique relates to the biochemistry of base determination and also implicates read length and read number, with consequences for read assembly. For example, the output from Sanger sequencing on a glass-capillary instrument provided by ABI is typically a small number of medium length (several hundred bases) chromatograms that are provisionally “called” (interpreted) as bases by software and presented visually for human verification. Long read sequencing (e.g., PACIFIC BIOSCIENCES, OXFORD NANOPORE) is meant to provide single or low numbers of much longer (>1,000) base reads. Short read sequencing (e.g., ILLUMINA) provides a large number (e.g., millions) of short reads (e.g., 50 or fewer bases) that are typically mapped to a reference and/or assembled de novo to show the original sequence. Illumina is accepted as an industry standard example of a next-generation sequencing (NGS) platform. Whatever instrument or technique is used, methods may include one or any combination of suitable “coverage” strategies, which involve determinations of what targets to sequence and at what coverage.

Coverage strategies may include, for example, transcriptome sequencing in which all RNA transcripts are sequenced redundantly, re-sequencing in which a presumptively very similar genome is known and only highly variable targets are sequenced, whole exome sequencing in which all expressed genes or exons are sequenced, or other coverage strategies. Even with a particular coverage strategy, one may opt for a certain depth of coverage. For example, for some applications, when NGS is used, 30× coverage is considered a standard coverage in which substantially all bases are sequenced redundantly such that each base, on average, appears in about 30 unique sequence reads. Certain preferred embodiments of the invention use low-pass whole genome sequencing (as used herein, “whole genome sequencing” means that a substantial portion such as at least 80% or 90% of a genome or at least a chromosome is sequenced). Low-pass whole genome sequencing (LP-WGS) is a technique in which each base in the entire genome is sequenced a few times (known as low-depth coverage) e.g., with a depth of coverage below about 5 and as low as 0.1-1 times. By reducing the depth of coverage, the cost of sequencing the whole genome is reduced while maintaining genome-scale coverage. LP-WGS is described in Christodoulou, 2023, Combined low-pass whole genome and targeted sequencing in liquid biopsies for pediatric solid tumors, NPJ Precision Onc 7:21 and Zheng, 2022, Experience of low-pass whole genome sequencing-based copy number variant analysis, Diagnostics (Basel) 12(5): 1098, both incorporated by reference.

Whatever technique and coverage are employed, methods include sequencing nucleic acid from a tumor. In certain preferred embodiments, LP-WGS is used to sequence substantially at least about 90% of a tumor genome at a coverage of about 15× or lower. The sequencing provides sequence data of the tumor nucleic acids. The sequence data may be analyzed to create a personalized tumor mutation profile, which includes any potential tumor variants and/or mutations. Low-pass sequencing is especially well suited for methods described herein where an objective is to detect SVs because, compared to other genotyping purposes, SVs are readily discoverable by LP-WGS as described herein.

A variety of different variants and mutations may be tracked using the tumor mutation profile. Typically, these variants are structural variants. Structural variants (SVs) are genomic abnormalities that may amplify, delete, or rearrange genomic regions of a tumor. It is possible and, in fact, common for more than one SV to occur in the same tumor. As used herein, an SV generally refers to a rearrangement, duplication, or deletion of a segment of length of at least about 1,000 bases. Methods of the disclosure may also be used to detect tumor-specific polymorphisms and/or small indels.

### Detection of Tumor-Specific Variants

The disclosure includes methods for analyzing sequence reads, as may be obtained from nucleic acid from tumors, to identify structural variants (SVs), and optionally filter out any putative structural variants that are not somatic (e.g., germline SVs or artifacts from sample processing or sequencing) to identify SVs that are specific to the tumor, i.e., tumor variants. Methods may include comparing tumor sequence to a reference by one or more algorithms, identifying structural variants in the tumor nucleic acid, and designing primers to specifically amplify those tumor variants. Sequence reads from tumor nucleic acid may first be cleaned up, mapped to a reference, and or subject to computational workflows to detect SVs.

Reads can be cleaned using known software methods such as fastp as described in Chen, et al., 2018, fastp: an ultra-fast all-in-one FASTQ preprocessor, Bioinformatics, 34(17):i884-i890, incorporated by reference. Cleaning may include trimming adapter sequences, removing low quality bases at the ends of reads and artifacts such as polyG tails. In some FFPE embodiments cleaning may include removing reads shorter than 30 bp instead of a standard 15 bp limit that may inadvertently select out shorter valid sequence reads resulting from sample fixation. Cleaned reads can be subjected to quality control using, for example, the FastQC available from the Babraham Institute, Cambridge UK.

Sequence reads, obtained via any known method, may be mapped to a reference using assembly and alignment techniques known in the art or developed for use in the workflow. Various strategies for the alignment and assembly of sequence reads, including the assembly of sequence reads into contigs, are described in detail in U.S. Pat. No. 8,209,130, incorporated by reference. Sequence assembly can be done by methods known in the art including reference-based assemblies, de novo assemblies, assembly by alignment, or combination methods. Sequence assembly is described in U.S. Pat. Nos. 8,165,821; 7,809,509; 6,223,128; U.S. Pub. 2011/0257889; and U.S. Pub. 2009/0318310, each incorporated by reference. Sequence assembly or mapping may employ assembly steps, alignment steps, or both. Assembly can be implemented, for example, by the program ‘The Short Sequence Assembly by k-mer search and 3′ read Extension’ (SSAKE), from Canada's Michael Smith Genome Sciences Centre (Vancouver, B.C., CA) (see, e.g., Warren et al., 2007, Assembling millions of short DNA sequences using SSAKE, Bioinformatics, 23:500-501, incorporated by reference). SSAKE cycles through a table of reads and searches a prefix tree for the longest possible overlap between any two sequences. SSAKE clusters reads into contigs.

In certain embodiments, reads are aligned to a reference human genome using Burrows-Wheeler Aligner version 0.5.7 for short alignments, and genotype calls are made using Genome Analysis Toolkit. See McKenna et al., 2010, The Genome Analysis Toolkit: a MapReduce framework for analyzing next-generation DNA sequencing data, Genome Res 20(9): 1297-1303, incorporated by reference (aka the GATK program). Reads may be assembled using SSAKE version 3.7. The resulting contiguous sequences (contigs) can be aligned to the reference (e.g., using BWA). In some embodiments, the reference genome may include GRCh38.

A workflow for SV detection from sequence reads and for primer design may be automated using tools such as Snakemake or Nextflow and custom programming using R or Python, for example, to link input/output across the various workflow steps. Some embodiments employ a computational pipeline that uses two or more different algorithms, each intended for finding SVs, to call putative SVs and merge the results. The computational pipeline may be used for mapping reads to a reference by a first algorithm (in a first mapping) and also by a second algorithm to identify SVs by each algorithm and then selecting the better result or merging the results of the multiple mapping steps to describe the structural variants. One of the algorithms may be a graph-based algorithm. In preferred embodiments, the first algorithm adds the reads to a genomic graph and finds a path through the graph best supported by the reads. This approach may be implemented by a suitable software platform such as the de Bruijn graph-based assembler GRIDSS. Methods may include software, tools, and techniques described in Cameron, 2017, GRIDSS: sensitive and specific genomic rearrangement detection using positional de Bruijn graph assembly, Genome Research 27(12):2050-2060 and Cameron, 2021, GRIDSS 2:
comprehensive characterization of somatic structural variation using single breakend variants structural variant phasing, Genome Biol 22(1):202, both incorporated by reference. In order to adapt to low-pass whole genome sequencing samples, variant calling parameters in the GRIDSS program may be changed including, for example, shortening the minimum length, minimum variant calling score, and minimum variant calling breakpoint quality and increasing the minimum variant calling size.

Preferably, the second algorithm aligns read pairs to a reference and searches for genomic regions in the reference where a significant number of read pairs align to the reference in positions inconsistent with an empirical insert size distribution for the read pairs. That algorithm may be implemented by a software platform such as BreakDancer. Methods may include software, tools, and techniques described in Chen, 2009, BreakDancer: an algorithm for high resolution mapping of genomic structural variation, Nat Methods 6(9):677-681, incorporated by reference. SplitSeq may be used to refine SV calls made by the first or second algorithm, especially those made with BreakDancer as described in Olsson, et al., 2015, Serial monitoring of circulating tumor DNA in patients with primary breast cancer for detection of occult metastatic disease, EMBO Mol Med, 7(8):1034-1047, incorporated herein by reference in its entirety. SplitSeq can be used to reconstruct the exact fusion sequence based on split reads and read pairs with one unmapped mate. Discordant reads can be re-aligned to reduce false positive SV calls. After merging of the SV calling paths using the first and second algorithms, the putative SVs can be annotated with genes that overlap SV breakpoints,

Methods may include filtering SVs that were identified by the mapping workflows to remove germline SVs and/or sample handling artefacts, thereby providing a set of somatic SVs, or tumor variants, present in the tumor DNA. The filtering step may involve comparing the putative SVs to at least one database of known germline SVs and removes matches from the putative SVs. It is understood that some of modern genomics is predicated on a view that there are sequenced and published “reference genomes” and that a sequencing genetic material from a subject gives data that can be analyzed by comparison to the reference. The language of variants sometimes refers to differences between the subject and the reference as a variant in the subject. From that perspective, many people may be born with benign germline SVs (relative to the reference). When sequencing DNA according to the embodiments herein, a variant calling pipeline may find those benign germline variants. Typically, one is more interested in somatic mutations that are specific to a tumor (from which the FFPE sample was created) as those may be used to specifically target and track tumor development, remission, and recurrence. Thus, for an MRD assay of the invention to achieve excellent sensitivity and specificity, all SVs found by sequencing are preferably filtered to remove benign germline variants from the putative set, leaving a set of tumor-specific somatic SVs. Filtering may include comparing to a database of known SVs to remove from consideration those that are documented to be benign. Such a database may include the Genome Aggregation Database (gnomAD) described in Chen, 2023, A genomic mutational constraint map using variation in 76,156 human genomes, Nature 625:92-100, incorporated by reference; Genome in a Bottle SVs described in Chapman, et al., 2020, A crowdsourced set of curated structural variants for the human genome, PLOS Comp Bio, 16(6): e1007933, incorporated by reference; or the database of human structural variation known as dbVar described in Lappalainen, 2013, DbVar and DGVa: public archives for genomic structural variation, Nucleic Acids Res 41(Databse Issue):D936-41, incorporated by reference.

The described workflows provide for mapping the sequence reads to a reference and identifying read mappings that indicate a structural variant in the tumor nucleic acid, relative to the non-tumor nucleic acid of the subject. That structural variant is tumor specific. It is a variant specific to the tumor, herein referred to as a tumor variant. Using methods of the disclosure, the tumor variant is found by sequencing tumor nucleic acid and analyzing the sequence data. A feature of the disclosure is that such a tumor variant may be confirmed by orthogonal testing. Thus, the invention provides methods for analyzing tumor nucleic acid from a tumor from a subject to discover one or more variants that are specific to the tumor and confirming by orthogonal testing that nucleic acid of the tumor harbors the variants and that the variants are specific to the tumor and thus useful as a tumor biomarker in an independent assay for the presence of the tumor in the subject.

### Selecting a Set of Marker Variants

Methods of the disclosure include a step of selecting from any and all detected tumor SVs a subset of those that constitute a set of marker variants to be used in a digital PCR MRD assay. Criteria for selecting the marker variants may include level of duplication; driver mutations; passenger mutations; truncal mutation; conservation across tumor clonality; suitability for PCR; other criteria; and any combination of the foregoing. The invention includes methods for ranking structural variants (SVs) and/or otherwise detecting and assigning relative ranks, in terms of selection for an MRD assay. Systematically ranking SVs provides an approach for the automatic selection of which SVs to interrogate in a diagnostic assay, such as a digital PCR assay for MRD from circulating-tumor DNA in blood or plasma. Methods include analyzing sequence data from tumor nucleic acid from a tumor of a subject to identify the presence and copy numbers of a plurality of tumor-specific structural variants (SVs) in the tumor nucleic acid compared to non-tumor nucleic acid from the subject; ranking the SVs wherein higher ranks are correlated to higher copy numbers; and providing reagents for an assay that detects a tumor signature comprising one or more of the SVs selected for having the higher ranks. Thus, methods may include determining copy number of detected SVs. Copy-number calling can then be performed to, for example, estimate tumor cell content in the sample and the degree to which the tumor genome may be rearranged. Genome-wide copy number information can be used later for prioritizing SVs for validation. Exemplary copy-number analysis can include ichorCNA described in Adalsteinsson, et al., 2017, Scalable whole-exome sequencing of cell-free DNA reveals high concordance with metastatic tumors, Nature Communications volume 8, Article number: 1324, incorporated herein by reference in its entirety.

Other criteria may also be used for selecting marker variants. For example, ranking may also include assigning a high rank to a truncal SV identified as an initiating truncal mutation of the tumor. The ranking step may include application of any other suitable criteria, such as the requirement for suitable primer binding sites by which one primer pair could amplify the multiple loci or instances of duplication of the marker variant. In certain tumor signature embodiments, the computer system is used to design multiple primer pairs that are useful to detect two or more of the SVs with higher ranks as a patient-specific, tumor-specific signature of the tumor in the subject.

### Assay Design

Methods may include designing and providing a plurality of copies of a primer pair that specifically amplify the sequence and storing the plurality of copies of a primer pair as reagents for use in one or more future assays for minimal residual disease. Designing the primer pair(s) may be implemented by a computer system. The computer system may also be used to design any or all other aspects of detection assay for the marker variant(s). The computer system may be used to design, for example, suitable fluorescent hydrolysis probes such as the probes sold under the trademark TAQMAN by Thermo Fisher Scientific (Waltham, MA). The computer system may be programmed to calculate and store conditions for a digital PCR (dPCR) assay such as: sample volume, dilution factor, partition number, reagent concentration, instrument settings, excitation and detection wavelengths, others, or any combination thereof. Output parameters from the computer system may be used as inputs to a suitable dPCR instrument or system, optionally carried from the assay design pipeline to the dPCR system by a direct data connection (e.g., WiFi or LAN) or by a managed system such as a laboratory information management system (LIMS).

From that, the computer system may thus provide conditions for an amplification reaction that will use a plurality of primer pairs designed to amplify a respective plurality of structural variants (SVs). The computer system output may also specific information such as sequences for the primer pairs that will amplify copies of the one or more SVs. Oligonucleotide reagents (primer pairs and fluorescent probes) maybe synthesized or obtained from a vendor such as Integrated DNA Technologies (Corralville, IA) and transferred (i.e., pipetted or dispensed) into reservoirs of the dPCR system. For example, the plurality of primer pairs are obtained as a reagent in one or more containers such as reagent tubes that are provisionally stored (e.g., lyophilized or in a freezer) and separately, subsequently dispensed to the detection assay instrument for use in the amplification reaction for detection of the plurality of SVs as a tumor-specific, patient specific signature of presences of the tumor.

### Detection Assay

Methods herein may include performing an assay to detect a marker variant in a sample from a subject; and reporting the presence of the tumor in the subject when the assay is positive for the marker variant in the sample.

The disclosed methods are useful for detection of any suitable target of interest in a sample. For example, methods are useful to detect nucleic acid from a pathogen in a mixed environmental sample or in a clinical sample that includes abundant host nucleic acid. The method may be used to detect fetal DNA in maternal blood or plasma. In certain preferred embodiments, the method is useful to detect a variant associated with a disease, such as an SV from tumor DNA, in cell-free DNA in a sample from a patient.

Any suitable sample may be used. For example, the sample may be blood, saliva, solid tissue, fine needle aspirate, a tumor biopsy (including material liberated from a formalin-fixed, paraffin embedded tumor sample), oral (e.g., buccal) swab, urine, stool, or any other sample. In certain embodiments, the sample comprises blood or plasma and the nucleic acid comprises cell-free DNA (cfDNA) in the blood or plasma.

Methods of the invention may be used to detect any nucleic acid feature of interest including, for example, specific sequences, genes, or variants (e.g., mutations), which may include polymorphisms, small indels, or structural variants (which may include deletions, rearrangements, large indels, translocations, copy number variants, or others). In certain embodiments, the variant is a structural variant (SV) and a pre-amplification is performed with a PCR primer pair design to anneal to sites that flank a breakpoint of the SV. Preferred embodiments provide methods useful to detect nucleic acid fragments containing targeted structural variants present in a sample at low abundance (e.g., as low as one copy).

In preferred liquid biopsy and dPCR for MRD embodiments, the obtaining step may involve receiving a blood collection tube or container containing blood or plasma that was obtained from the subject via blood draw. The sample may include cell-free DNA from blood or plasma from the subject. The sample may be less than about 100 mL of blood or plasma and wherein the cell-free DNA is present at a concentration between about 1 and 50 ng/mL or lower in the blood or plasma circulating in the subject.

In certain optional in vitro “pre amplification” embodiment, the detection may proceed by at least two distinct stages or mechanisms that include (i) copying the marker variant using variant-specific primers and tailed primers to form tailed amplicons and (ii) amplifying the tailed amplicons in the presence of probes that indicate the presence of amplicons from the target of interest in the aqueous compartment. In some embodiments, a pre-amplification step may use primers designed to specifically amplify the marker variant. For example, if the variant is a structural variant, the pre-amplification may use a pair of primers designed to anneal to nucleic acid at locations that flank a breakpoint of the structural variant. This strategy further enriches the sample for copies of the marker variant, ensuring that the presence of the variant is detected in the subsequent detection steps. This pre-amplification step specifically addresses problems associated with some dead volume of sample that resists detection by existing digital PCR (dPCR) approaches. Due to the stochastic nature of sampling, some very minor fraction of a sample will, by chance, typically go undetected by dPCR. Here, the pre-amplification step may increase quantity of the marker variant prior to the partitioning and dPCR detection, reducing the likelihood that the target of interest will be undetected due to stochastic loss in dead volume. After the pre-amplification, the sample may be partitioned into aqueous compartments.

Regardless of any optional in vitro “pre-amplification” embodiments, the detection assay is useful to detect the tumor, and may be used as an MRD assay.

### Limit of Detection

Methods may be used in assays for minimal residual disease (MRD), e.g., after a treatment to eradicate the tumor. Methods my include obtaining a sample from a subject who has undergone treatment for a tumor; performing an amplification reaction in the sample using a primer pair that is designed to amplify a tumor-specific marker variant. Methods for detecting minimal residual disease (MRD) may include performing an MRD assay on a sample from a subject who has undergone cancer treatment to detect a set of selected marker variants that are in tumor nucleic acid and not in non-tumor nucleic acid of the subject; and reporting the presence of MRD in the subject when the assay is positive for at least one the marker variants in the sample, wherein the MRD is detected with sensitivity <10 ppm, and specificity >99.5%. The sensitivity and specificity may be achieved using fewer than thirty (e.g., 16) of the marker variants. It has been found that as few as sixteen (16) marker variants may be used in a digital PCR assay and while still obtaining sensitivity better than 10 ppm and specificity is greater than 99.5%. In fact, using methods of the invention sensitivity of 0.3 ppm was obtained. Thus, methods of the invention provide for very highly sensitive (i.e., very low LoD) and specific assays for MRD.

Embodiments of the invention provide an MRD assay with a limit of blank of zero and a sensitivity of <10 ppm. It has been found that the limit of blank may be zero in that the detection of any PCR-positive partition may be treated as a positive detection of MRD and using feature of the disclosure including fewer than 30, e.g., 16, marker variants, MRD detection is reliable at a limit of blank of zero and a sensitivity of <10 ppm including, actually a sensitivity of 0.3 ppm. To summarize, the invention provides an MRD assay with a sensitivity (LoD95) of <10PPM and a specificity of >99.5%. This is by performing the MRD result using structural variants and/or dPCR. Other factors include using a WGS sequencing at <30× (preferably about 15×) to identify the variants for tracking. The disclosed assay operates with a turn-around time from blood sample to result of <4 days. The assay has a limit of blank of 0 and an MRD positive result is reported for any signal above 0. The LoD <10PPM is obtained by tracking only 16 variants, where others in the field are tracking >1000 variant to achieve a comparable LOD. Assays of the disclosure detect down to 0.3 PPM, 10× fold lower than an LOD 95. Thus, the disclosure provides an MRD assay with a lower limit of detection at least 10× lower than that of the LoD 95.

### Digital PCR

The described detection assay may be any suitable assay including, for example, nucleic acid sequencing, DNA microarray analysis, fluorescent in situ hybridization, PCR, quantitative PCR, or digital PCR (dPCR). In preferred embodiments, the detection assay is dPCR and the sample comprises blood or plasma from the subject and the assay analyzes cell-free nucleic acid in the blood or plasma. For the assay, dPCR may include partitioning the sample into aqueous partitions that include PCR reagents and fluorescent probes for the amplicons and conducting the amplification reaction in the aqueous partitions. The assay comprises performing an amplification reaction to detect amplification of the copies of the one or more SVs.

The assay includes partitioning the sample into aqueous partitions and performing an amplification reaction in the aqueous partitions using at least one primer specific for the sequence and a probe that provides a signal when the amplification reaction using at least one primer generates an amplification product. The method may include partitioning the sample into aqueous partitions that include PCR reagents and fluorescent probes for the amplicons, conducting the amplification reaction in the aqueous partitions, and detecting fluorescence from the partitions to detect the residual presence of the tumor after the treatment. Those dPCR steps may all be automated and/or performed using a commercially available dPCR instrument or system. The dPCR system may detect fluorescence from the partitions and provide output indicating a number of partitions that include the marker variant.

For a subject in whom a tumor has been diagnosed, and a sample of the tumor (biopsy, FFPE slice) obtained, a treatment may have been administered to eradicate the tumor. For example, the person may have undergone surgical resection to remove the tumor, radiation therapy to ablate the tumor, or chemotherapy to kill cells of the tumor. The person may spend some amount of time feeling the benefit of the treatment, living a cancer-free life. However, an insidious aspect of cancer is that, even after treatment to eradicate a tumor, that cancer may return, later, e.g., months or even years later. For a cancer that does return, because some vanishingly small number of cells escaped eradication by the treatment (i.e., MRD), there may still be the opportunity to kill those cells and cure the cancer if the presence of that MRD is detected in good time. In such situations, the invention provides methods that provide a detection assay that can detect MRD even when other biomarkers are expected to be beyond the LoD for the assay. Methods of the invention may be used to provide the elements of a detection assay (e.g., assay design and reagents such as PCR primers and detection probes) that may be kept and used repeatedly over time, e.g., tens of times or more, over months and years, conveniently and inexpensively. The detection assay may be a PCR-based assay that only needs a blood draw, as described, so that, after undergoing treatment, a person may know via a relatively quick, inexpensive, and minimally invasive test, whether there is any evidence of MRD.

The invention provides an assay that uses structural variants (SVs) as markers for tumor DNA. After tumor DNA is analyzed for tumor variants, tumor variant-specific primers are used in digital PCR to interrogate a sample from the patient for the presence of tumor DNA as an assay for MRD. Using as few as 16 SVs as tumor markers, digital PCR assays according to the disclosure have a limit of detection (LoD), or sensitivity, of less than 10 parts per million (ppm) and a specificity greater than 99.5%.

What is claimed is:

1. A method comprising:
analyzing sequence data from tumor nucleic acid from a tumor from a subject to identify tumor-specific variants that are in the tumor nucleic acid and that are not in non-tumor nucleic acid of the subject;
selecting, from among the plurality of tumor-specific variants, a set of marker variants;
performing an assay to detect the marker variants in a sample from the subject; and
reporting the presence of tumor DNA in the subject when the assay is positive for at least one the marker variants in the sample, wherein a limit of detection (sensitivity) of the tumor DNA in the sample is less than 10 parts per million.

2. The method of claim 1, further comprising obtaining the sequence data by performing low-pass whole genome sequencing (lpWGS) on the tumor nucleic acid, wherein lpWGS means sequencing with an average coverage less than about 20× coverage.

3. The method of claim 1, wherein the set of marker variants includes fewer than 50 selected marker variants.

4. The method of claim 1, wherein a specificity of the detection of the tumor DNA in the sample is >99.5%.

5. The method of claim 6, wherein the assay is performed after the subject has been treated to remove the tumor, and the reporting includes reporting the presence of minimal residual disease (MRD) at the sensitivity <10 ppm and the specificity >99.5.

6. The method of claim 7, wherein the set of marker variants includes fewer than 20 selected marker variants.

7. The method of claim 1, wherein the assay comprises digital PCR.

8. The method of claim 7, wherein method include subjecting the sample to a pre-amplification step prior to detecting the marker variants.

9. The method of claim 8, wherein the pre-amplification step comprises a linear amplification and wherein the detecting of the marker variants by the digital PCR includes an exponential amplification reaction.

10. The method of claim 7, set of marker variants includes between 3 and 20 marker variants, and wherein the digital PCR is used to detect greater than 3 of marker variants in two color channels at a time via radial multiplexing.

11. The method of claim 1, wherein the digital PCR is performed with a limit of blank of zero and the presence of tumor DNA is reported if a positive amplification result is detected in any number of digital PCR partitions greater than zero.

12. The method of claim 1, wherein the marker variants comprise structural variants (SVs) or the breakpoints of the SVs.

13. The method of claim 1, further comprising—after the tumor-specific variants are identified—providing PCR primers that are specifically designed to amplify only the tumor-specific variants.

14. The method of claim 1, wherein the assay comprises digital PCR performed in aqueous partitions, wherein the aqueous partitions comprise droplets or wells, wherein the marker variants comprise SVs, and wherein the marker variants are detected by the digital PCR using fluorescent probes.

15. The method of claim 1, wherein the sample from the subject comprises blood or plasma obtained by a blood draw from the subject.

16. The method of claim 1, wherein the limit of detection (sensitivity) of the tumor DNA in the sample is 0.3 ppm.
